(19) United States
(12) Patent Application Publication
(10) Pub. No.: US 2024/0398401 A1
(43) Pub. Date: Dec. 5, 2024

(54) Suture Locking Device

(71) Applicant: Stitchlock, Inc., Webster, TX (US)

(72) Inventors: Clayton L. Moliver, Houston, TX (US); Gerald Baty, The Woodlands, TX (US); Mark Mathew Gittleman, Houston, TX (US)

(73) Assignee: Stitchlock, Inc., Webster, TX (US)

(21) Appl. No.: 18/678,069

(22) Filed: May 30, 2024

Related U.S. Application Data
(60) Provisional application No. 63/504,892, filed on May 30, 2023.

(51) Int. Cl. A61B 17/04; A61B 90/92

(52) U.S. Cl. CPC A61B 17/0401; A61B 90/92; A61B 2017/0414

(57) ABSTRACT

Provided herein are devices for locking a suture in place on a wound without the need of tying a knot. The device has a substantially rectangular or circular shape with at least one aperture therethrough and at least one groove disposed across the device and connecting with the at least one aperture. The shape, dimensions and/or polymer content of the groove and the roughened inner surface or the teeth or flexible fingers disposed on the side surfaces of the grooves enables securement or binding of the suture. The devices may have secondary features that further tighten the suture.

### CROSS-REFERENCE TO RELATED APPLICATIONS

This non-provisional application claims benefit of priority under 35 U.S.C. § 119(e) of provisional application U.S. Ser. No. 63/504,892, filed May 30, 2023, the entirety of which is hereby incorporated by reference.

### BACKGROUND OF THE INVENTION

### Field of the Invention

The present invention relates generally to the fields of medicine, wound closures and sutures. More specifically, the present invention relates to devices for locking sutures while stitching a wound closed.

### Description of the Related Art

Wound closure modalities vary with the type of wound, depth, tissue type to be closed, degree of tension required, and cosmetic results desired. Wounds are typically closed with one or more of staples, sutures, or adhesives.

Sutures are the most commonly employed closure modality and suture threads have been made from many materials, including bioabsorbable materials that ultimately completely break down in the body or non-absorbable materials that are permanent and non-degradable. Sutures may be threaded through a separate needle, but typically consist of a suture thread attached to the needle. Non-absorbable sutures are generally utilized where greater tensile strength is required. Non-absorbable sutures also are used in wounds requiring prolonged wound healing and prolonged physical support. Absorbable sutures are utilized where suture removal might affect the repair or where long-term physical support is unnecessary after complete wound healing. If required, a double layer closure with absorbable sutures can be employed to increase tensile strength. In superficial wound closure, absorbable sutures may be used where decreased tension is permissible and may provide better wound edge approximation and aesthetic outcome. A wide variety of surgical needles are available. The shape and size of the needle body and the configuration of the needle tip is typically selected based upon the needs of the particular application.

Various techniques for suture application have been long known and are selected based on the type of wound, surgical situation, tension required and desired cosmetic outcome. For rapid control of bleeding or long wounds, running sutures may be used with the result that tension is spread along the length of the wound. However, with the running stitch, failure of the stitch or infection may result in opening of the entire wound. For both subcutaneous and superficial closure, interrupted sutures permit a close approximation of the skin and fascia with less risk of impaired cutaneous circulation. In the event of infection, partial wound repair is possible. However, interrupted sutures require repeated knot tying which requires time, training, and considerable dexterity.

In addition to the labor intensity and time expended on knot tying, complications associated with knot tying include 1) spitting, where the knotted suture pushes through the skin after a subcutaneous closure, 2) a greater nidus for infection in the knot interstices, 3) increased mass of the knot resulting in greater bulk foreign body irritation and scarring, and 4) slippage with loosening or loss of the knot. Suture loops associated with knot tying may lead to ischemia from strangulated tissue and increased risk of dehiscence or rupture at the surgical wound.

One solution to the problems associated with knot tying is through the use of barbed sutures or self-retaining sutures, which were first disclosed in U.S. Pat. No. 3,123,077. Additional patents on barbed sutures include U.S. Pat. No. 5,374,268 to armed anchors having barb-like projections, U.S. Pat. Nos. 5,584,859 and 6,264,675 to suture assemblies having barbed lateral members, and U.S. Pat. Nos. 5,931,855 and 6,241,747 to bidirectional barbed sutures. U.S. Pat. Nos. 8,795,332 and 8,915,943 disclose other types of barbed sutures, including staggered barbs, and the manufacture of barbed sutures.

The first end loop suture patent, U.S. Pat. No. 5,100,421 describes introducing the needle through the tissue and pulling the suture thread all the way through the tissue to a large terminal loop. The needle is then run through the terminal loop several times and finally the suture thread is pulled tight enough through the loop to approximate the tissue surfaces to be closed. The suture thread is then introduced continuously between opposing tissue surfaces until the wound is closed. The patent suggests that circular tissues such as bowel walls can be effectively sutured in this manner. The problem with running a continuous running suture around a tubular tissue such as, for example, a blood vessel, duct, bowel or esophagus, is that the suture can form a circular constricture around the tubular tissue thus restricting the normal movement of the tissue.

U.S. Pat. No. 10,016,196 describes a barbed suture with a terminal loop element designed and dimensioned to form an end loop through which the suture thread is pulled and secured to the loop by knotting or tying. Similarly, U.S. Pat. Nos. 10,548,592 and 10,492,780 describe certain barbed sutures having terminal loops where the suture is passed back through the loop to form an anchor for a running stitch. The barbs on the suture material are intended to prevent backward slippage through tissue. However, in these prior patents the terminal loops are not dimensioned to lock the barbed suture against backward movement through the loop.

Since their introduction, barbed sutures have been utilized in various surgical applications and are available in both absorbable and non-absorbable monofilament materials as well as in braided form. The first barbed suture was described in U.S. Pat. No. 3,123,077. Today, widely utilized barbed sutures include the Quill SRS (Quill Self-Retaining System; Angiotech Pharmaceuticals, Vancouver, British Columbia, Canada), which has bidirectional barbs, the V-Loc Absorbable Wound Closure device (Covidien, Mansfield, MA, USA), which has unidirectional barbs with 1 needle and a loop at the end; and the Stratafix (STRATAFIX Knotless Tissue Control Devices, Ethicon Inc., Somerville, NJ, USA), which presents a spiral distribution of the barbs and anchors. Despite the advantages of barbed sutures, sufficient strength in the closure to prevent dehiscence has remained elusive.

Thus, there remain unmet needs in the art for closing the loop of a placed suture without the need for tying a knot. Particularly, the art is deficient in a separate device that attaches to the suture and provides a strong reliable self-retaining interrupted suturing that can be quickly implemented. The present invention fulfils this longstanding need and desire in the art.

### SUMMARY OF THE INVENTION

The present invention is directed to a device for locking a suture on a wound. The body has a substantially geometrical shape and at least one aperture disposed through the device. At least one groove is disposed across a top side of the body on either side of the aperture. The present invention is directed to a related device that further comprises at least one open-ended slot formed longitudinally into the body proximate to each of the at least one groove on either side of the aperture, said slot having a perpendicular opening through which the suture is received and tightened.

The present invention is further directed to a stitch locking system. The system comprises the device for locking a suture described herein and a suture attached by an end thereof to a distal end of the groove.

The present invention is directed further to a related device for locking a suture on a wound. The device has a substantially rectangular body made of an absorbable polymer or a nonabsorbable polymer, where the body has a convex top side and an underside with a rounded perimeter thereon, a central aperture therethrough and a substantially rectangular groove disposed longitudinally across the top side on either side of the central aperture. The central aperture comprises a circumferential curvature and the groove comprises a curved shoulder along one upper edge or both upper edges.

The present invention is directed further to a related device for locking a suture on a wound. The device has a circular body comprising an absorbable polymer or a nonabsorbable polymer, where the body has a top side and an underside with a rounded circumferential edge, a central aperture therethrough and a first V-shaped groove disposed longitudinally across the top side on either side of the central aperture. The central aperture comprises a circumferential curvature and the first V-shaped groove comprises a curved shoulder along one upper edge or both upper edges.

The present invention is directed further still to a device for locking a suture on a wound. The device has a body with a substantially circular perimeter, a substantially flat upper surface and a substantially flat lower surface. An aperture is disposed perpendicularly through the center of the device. A first groove and a second groove each are disposed across the upper surface of the body on either side of the aperture, where the first groove and the second groove comprise means for increasing securement of the suture disposed on side surfaces thereof. The present invention is directed to a related device that further comprises a secondary slot with an open end at the circular perimeter and extending longitudinally into the body proximate to the first groove and to the second groove on either side of the aperture, where the secondary slot has a perpendicular opening through which the suture is received and tightened.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1C are views of a first embodiment of the STITCHLOCK device without a suture (FIG. 1A), with the suture positioned within the groove (FIG. 1B) and with a suture positioned around the curvature of the aperture (FIGS. 1B-1C).

FIGS. 2A-2E illustrate how a suture is positioned in the STITCHLOCK device of FIG. 1A. FIG. 2A shows the positioning of the suture proximate to the groove and around the aperture of the STITCHLOCK device prior to locking it in the groove and FIG. 2B shows the suture within the groove and around the aperture. FIG. 2C is a cross-sectional view showing the curvature of the aperture. FIG. 2D illustrates how the suture is compressed within the groove to lock it in place. FIG. 2E shows the relative decrease in diameter of the suture after it is locked within the device shown in FIGS. 2A-2C.

FIG. 3 is a cartoon of a wound closing with the STITCHLOCK device.

FIGS. 4A-4B are views of a second embodiment of the STITCHLOCK device showing the overall circular circumference, flat underside and convex top side (FIG. 4A) and the curvature along the aperture (FIG. 4B).

FIGS. 5A-5B are views of a third embodiment of the STITCHLOCK device showing the overall circular circumference and the convex underside and convex top side (FIG. 5A) and the curvature along the aperture (FIG. 5B).

FIG. 6 is a view of a fourth embodiment of the STITCHLOCK device differing from the device illustrated in FIGS. 5A-5B in that first and second grooves are positioned 90 degrees relative to each other.

FIGS. 7A-7C illustrate a fifth embodiment of the STITCHLOCK device in cross-section where the groove has shoulders at the upper edge. FIG. 7A shows the suture prior to placement within the groove. FIG. 7B illustrates how the shoulders impinge on the suture during placement. FIG. 7C illustrates how the suture is compressed to maintain its placement within the groove.

FIGS. 8A-8E illustrate a sixth embodiment of the STITCHLOCK device in cross-section where the groove comprises a soft or absorbable polymer therein to secure a suture that has a diameter less than or equal to that of the groove.

FIGS. 9A-9B illustrate a seventh embodiment of the STITCHLOCK device in cross-section where the groove is V-shaped (FIG. 9A) and engages with the suture at the bottom of the groove (FIG. 9B).

FIGS. 10A-10B illustrate an eighth embodiment of the STITCHLOCK device in cross-section where the groove is V-shaped with one shoulder (FIG. 9A). FIG. 9B illustrates how the combination of the V-shape and the shoulder compress the suture to secure it within the groove.

FIGS. 11A-11B illustrate a ninth embodiment of the STITCHLOCK device in cross-section where the groove is V-shaped with two shoulders (FIG. 11A). FIG. 11B illustrates how the combination of the V-shape and the shoulder compress the suture to secure it within the groove.

FIGS. 12A-12B show a 3-dimensional section of the two-shouldered V-shaped groove of the STITCHLOCK device of FIG. 11A (FIG. 12A) in an enlarged cross-sectional view (FIG. 12B).

FIGS. 13A-13B illustrate a tenth embodiment of the STITCHLOCK device in cross-section where the surface of the groove is roughened (FIG. 13A) where a combination of friction, resistance and compression secure the suture within the groove (FIG. 13B).

FIG. 14 illustrates an eleventh embodiment of the STITCHLOCK device shown in an isometric view where a plurality of teeth with substantially sharp ends are disposed on the surfaces of the first groove and on the second groove to extend interiorly therein.

FIG. 15 illustrates a twelfth embodiment of the STITCHLOCK device similar to FIG. 14 a plurality of secondary slots designed to move and squeeze the suture.

FIG. 16 illustrates a thirteenth embodiment of the STITCHLOCK device overall structurally similar to the device of FIG. 14 where a plurality of fingers are disposed on the surfaces of the first groove and on the second groove to extend interiorly and to angle outwardly to bind the suture and prevent it from being pulled out.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein “another” or “other” may mean at least a second or more of the same or different claim element or components thereof.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “includes” or “including” is used herein to mean “including, but not limited to”. The terms “includes”, “including” and “including but not limited to” are used interchangeably.

As used herein, the term “about” refers to a numeric value, including, for example, whole numbers, fractions, and percentages, whether or not explicitly indicated. The term “about” generally refers to a range of numerical values (e.g., +5-10% of the recited value) that one of ordinary skill in the art would consider equivalent to the recited value (e.g., having the same function or result).

As used herein, the terms “STITCHLOCK device” and “device”, “groove” and “slot” and “central aperture” and “aperture” are interchangeable.

As used herein, the term “wound” refers to an incision made during a surgical procedure or to an injury to living tissue that requires closing with sutures for proper healing.

As used herein, the terms proximal and distal refer to the ends of a suture nearest to or farthest from the needle end, respectively.

As used herein, the ordinal adjectives “first” and “second” unless otherwise specified are used to describe a common object, merely indicate that different instances of like objects are being referred to, and are not intended to imply that the objects so described must be in a given sequence, either temporally, spatially, in ranking, or in any other manner.

As used herein, the term “substantially sharp” refers to an end or edge that is sufficient to embed itself in the suture without cutting or severing the suture.

As used herein, the term “secondary slot” refers to an optional groove or notch extending longitudinally into the device body and formed with a perpendicular opening therethrough through which the suture is drawn and further secured to the device.

In one embodiment of the present invention, there is provided a device for locking a suture on a wound, comprising a body with a substantially geometrical shape; at least one aperture disposed through the device; and at least one groove disposed across a top side of the body on either side of the aperture. Further to this embodiment, the device comprises at least one open-ended slot formed longitudinally into the body proximate to each of the at least one groove on either side of the aperture, said slot having a perpendicular opening through which the suture is received and tightened.

In both embodiments, the body may comprise a substantially rectangular shape or a circular shape. Also, in both embodiments, the body may comprise rounded edges at a perimeter or a circumference thereon. In one aspect of this embodiment, the body may comprise a convex top side and a flat underside. In another aspect the body may comprise a convex upper surface and a convex lower surface. In yet another aspect, the aperture may be disposed through a center of the device. In addition to both embodiment and all aspects thereof, the body may be made of an absorbable or non-absorbable polymer. Furthermore, the body may be color-coded or numbered to correlate to a suture size or suture type or a combination thereof. Furthermore, the aperture may be curved at a circumference thereof.

In another aspect of both embodiments, the device may comprise one groove. In yet another aspect, the device may comprise a pair of grooves disposed at 90 degrees relative to one another. In yet another embodiment, the device may comprise a plurality of grooves radially disposed on either side of the aperture. In addition, in yet another aspect of these embodiments, the groove may have a diameter sufficient to secure therein a suture of a specific size. In yet another aspect, the groove may have a diameter greater than a diameter of a suture, where the groove contains a soft, absorbable polymer. In yet another aspect, the groove may narrow across a width thereof from the aperture to an edge of the device or may narrow across the width from the edge of the device to the aperture. Furthermore, the groove may comprise at least one roughened inner surface thereof or a plurality of flexible fingers or a plurality of teeth with substantially sharp ends disposed on at least one inner surface thereof. In all embodiments and aspects thereof, the groove may have a rectangular shape, may have a U-shape or may have a V-shape. Also in all embodiments the body may be color-coded or numbered to correlate to a suture size or suture type or a combination thereof.

In another embodiment of the present invention, there is provided a stitch locking system, comprising the device as described supra; and a suture attached by an end thereof to a distal end of the groove.

In yet another embodiment of the present invention, there is provided a device for locking a suture on a wound, comprising a substantially rectangular body made of an absorbable polymer or a nonabsorbable polymer, where the body has a convex top side and an underside with a rounded perimeter thereon, a central aperture therethrough and a substantially rectangular groove disposed longitudinally across the top side on either side of the central aperture; where the central aperture comprises a circumferential curvature and the groove comprises a curved shoulder along one upper edge or both upper edges.

In this embodiment, the underside may be flat or convex. Further in this embodiment, the groove may comprise at least one roughened inner surface. In one aspect of this embodiment, the substantially rectangular groove may have a diameter greater than a diameter of each of a plurality of a suture sizes, where the substantially rectangular groove containing a soft, absorbable polymer therein. In another aspect, the groove may have a diameter sufficient to secure therein a suture of a specific size.

In yet another embodiment of the present invention, there is provided a device for locking a suture on a wound, comprising a circular body comprising an absorbable polymer or a nonabsorbable polymer, where the body has a top side and an underside with a rounded circumferential edge, a central aperture therethrough and a first V-shaped groove disposed longitudinally across the top side on either side of the central aperture; where the central aperture comprising a circumferential curvature and the first V-shaped groove comprising a curved shoulder along one upper edge or both upper edges. Further to this embodiment, the device comprises a second V-shaped groove disposed across the top side on either side of the central aperture at 90 degrees relative to the first V-shaped groove.

In one aspect of both embodiments the first V-shaped groove and the second V-shaped groove each may have a same dimension or have different dimensions each sufficient to secure therein a suture of a specific size therein. In another aspect of both embodiments the first V-shaped groove or the second V-shaped groove or a combination thereof may have a diameter greater than a diameter of a plurality of suture sizes and a soft, absorbable polymer contained therein. In both embodiments, the underside may be flat or convex.

In yet another embodiment of the present invention, there is provided a device for locking a suture on a wound, comprising a body with a substantially circular perimeter, a substantially flat upper surface and a substantially flat lower surface; an aperture disposed perpendicularly through the center of the device; a first groove and a second groove each disposed across the upper surface of the body on either side of the aperture, where the first groove and the second groove comprise means for increasing securement of the suture disposed on side surfaces thereof. Further to this embodiment the device comprises a secondary slot with an open end at the circular perimeter and extending longitudinally into the body proximate to the first groove and to the second groove on either side of the aperture, where the secondary slot having a perpendicular opening through which the suture is received and tightened.

In one aspect of both embodiments, the means for increasing securement of the suture may comprise a plurality of teeth with sharp ends extending interiorly from the side surfaces of the first groove and the second groove. In another aspect of both embodiments, the means for increasing securement of the suture may comprise a plurality of flexible fingers extending laterally into the interiors of the first and second grooves and angling outwardly toward the circular perimeter. Also in yet another aspect of both embodiments, the first groove and the second groove may have dimensions sufficient to secure a suture of a single size therein. In yet another aspect of both embodiments the first groove and the second groove may have dimensions sufficient to secure sutures of different sizes therein. In both embodiments and all aspects thereof the first groove and the second groove independently may have a rectangular shape, a U-shape or a V-shape.

Provided herein are devices or STITCHLOCK devices for locking a suture in position on a wound without tying a knot. The devices are unique and separate from needles or sutures and are designed to attach the suture end opposite from the needle end. The sutures may be mechanically or chemically attached at the time of suturing or packaged with the device already attached at the end of a suture. A non-limiting example of attachment is gluing. Each STITCHLOCK device is engineered with thicknesses, width or depth or a combination thereof to secure only a specific, standard thickness or diameter of suture as is known in the art and defined by standard industry nomenclature, USP, or, alternatively, may be engineered with dimensions to secure a suture of any thickness or diameter or with dimensions to secure a barbed suture or may be engineered to secure more than one suture type. In non-limiting examples, the device may comprise either a non-absorbable material or an absorbable material in two or more sizes each able to secure two or more different thicknesses of suture. The devices may be color coded or sequentially numbered to correlate to the different size and/or types of sutures to which they will connect.

The devices are made with standard types of medical grade absorbable or nonabsorbable polymers as are known and standard in the art. The device may be manufactured, fabricated or produced by known means. For example, but not limited to, the device may be made by cutting them from a solid block of polymer with a laser, by injection molding, by 3D printing, or by chemical etching. In certain embodiments the device has drug-eluting capabilities, such as, but not limited to, an antibiotic, as the drugs are embedded in the medical grade polymers.

The device may be substantially rectangular or circular in shape with a flat or convex underside and a convex top side. The device has dimensions of about 3 mm to about 20 mm in length, about 2 mm to about 18 mm in width and about 2 mm to about 8 mm in thickness. The device comprises at least one aperture with a diameter of about 1 mm to about 5 mm. In one non-limiting example, the aperture may be a central aperture that is circular and about 3 mm in diameter. The device has no sharp edges around the perimeter or at the edges of the central aperture. The curvature of the aperture enables the suture to stay wrapped around the opening, without the walls acting as a fulcrum to cut the suture.

A longitudinal groove or slot extends down the middle of the long axis of the device and extends around the device at its distal edges and down into an aperture, curving around partially to the back of the device. The groove or slot has a depth of about 0.05 mm to about 3 mm. For example, an average depth of the groove may be about 1.5 mm. Alternatively, the device may comprise more than one groove, for example, but not limited to, a pair of grooves or slots at 90 degrees to one another or a plurality of grooves disposed radially across the aperture. In cross-section the groove may be rectangular or rounded at the bottom, such as in a U-shape, or may be V-shaped in cross-section being wider at the surface and without any width at the depth thereof. In a general non-limiting example, the groove narrows across the width thereof as it progresses from the central aperture to the edge of the device or from the edge of the device to the central aperture including, in the case of the V-shaped groove, narrowing towards the bottom tapered end of the groove

The groove may comprise a longitudinal shoulder or ridge on one or both sides thereof that extends about half way down the wall of the groove thereby allowing a haptic response as the suture is delivered past the ridge into the depth of the groove. This completes the locking mechanism of the device and obviates the need for tying a knot. The groove may contain a polymer, for example, an absorbable polymer softer than that comprising the device, which enables locking of sutures with diameters less than that of the groove. Alternatively, the inner surfaces of the groove may be roughened, or may comprise means for increasing securement of the suture within the groove, for example, but not limited to, a plurality of teeth, optionally with substantially sharp ends or tips, or a plurality of flexible fingers all of which extending into the groove and all of which in addition to compression of the suture within the groove reduces slippage via added friction, application of reverse pressure to the suture or mechanical binding of the suture.

During use, such as during a wound closure, the device enables a suture to be passed from below, through the central aperture and down one of the grooves. The distal end of the suture, furthest from the needle end, is thus captured and secured or locked within the depth of the groove and held there by the surface tension of the length of the groove and the curvature of the groove as it circumvents the central aperture. The needle is then passed downward through the tissues to be approximated and back up again through the same opening in the device. The tissues to be approximated are pulled tight and the suture is layer down into the groove on the opposite side of the first pass of the suture, thus completing the loop without the need for tying a knot.

Alternatively, to aid the suture in locking itself into the groove, a secondary slot, groove or notch with a perpendicular opening therethrough may be formed proximate to the first and second grooves, but do not extend to the central aperture. Basically, the suture goes from top down through the central aperture in the device, into the wound to close it, then up through the central aperture and is locked into the first or second grooves as appropriate both inbound and outbound. The secondary slots are positioned so that the suture may be wrapped around the bottom of the device and then pulled up therethrough and along the slot toward the central aperture as a secondary lock. The secondary slot optionally may have internal features as with the first and second grooves, but they are disposed or formed horizontally rather than vertically, as in the first and second grooves, since the suture is perpendicular within the secondary slot.

Particularly, embodiments of the present invention are better illustrated with reference to the Figure(s), however, such reference is not meant to limit the present invention in any fashion. The embodiments and variations described in detail herein are to be interpreted by the appended claims and equivalents thereof.

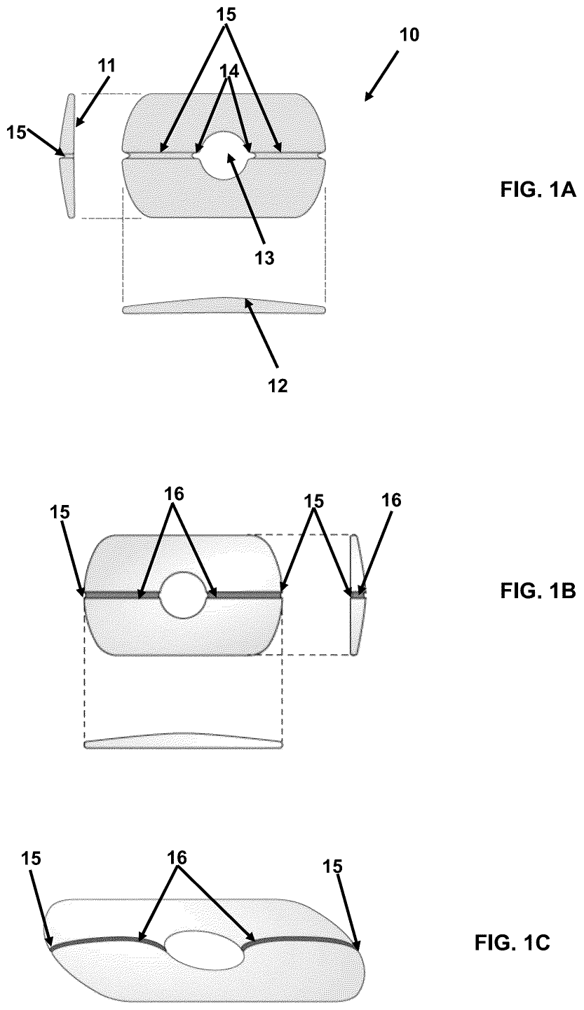

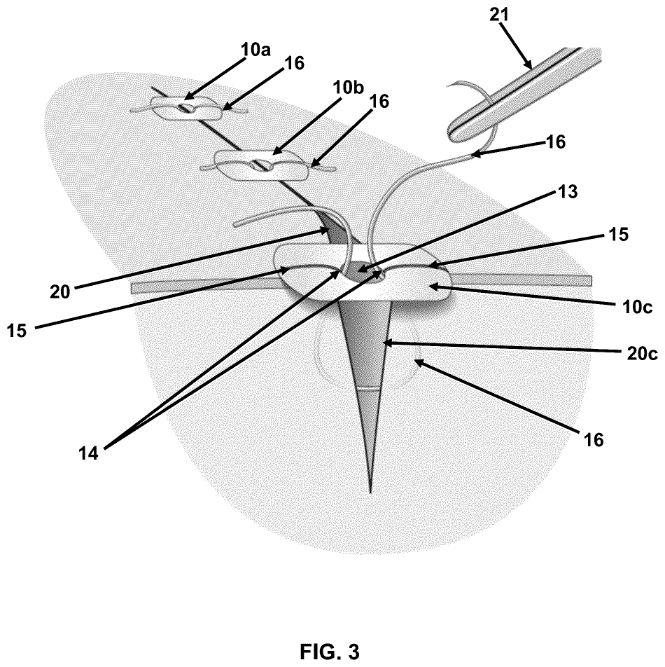

FIG. 1A illustrates one embodiment of the STITCHLOCK device 10. The device is substantially rectangular and has a flat underside 11 and a curved top side 12 with no sharp edges and a central aperture 13 with a curvature shown at 14. A groove 15 is formed across the top side and joins with the aperture. The central aperture enables passage of the suture 16 and needle (not shown) (see FIG. 3). The flat underside enables the device to be grasped during placement of the suture. The dimensions of the groove are designed to enable the suture to engage around the curvature of the aperture and down the length of the groove such that the suture is compressed about 10% to about 60% of its original diameter. With continued reference to FIG. 1A, FIGS. 1A-1B show the suture 16 engaged within the groove 15.

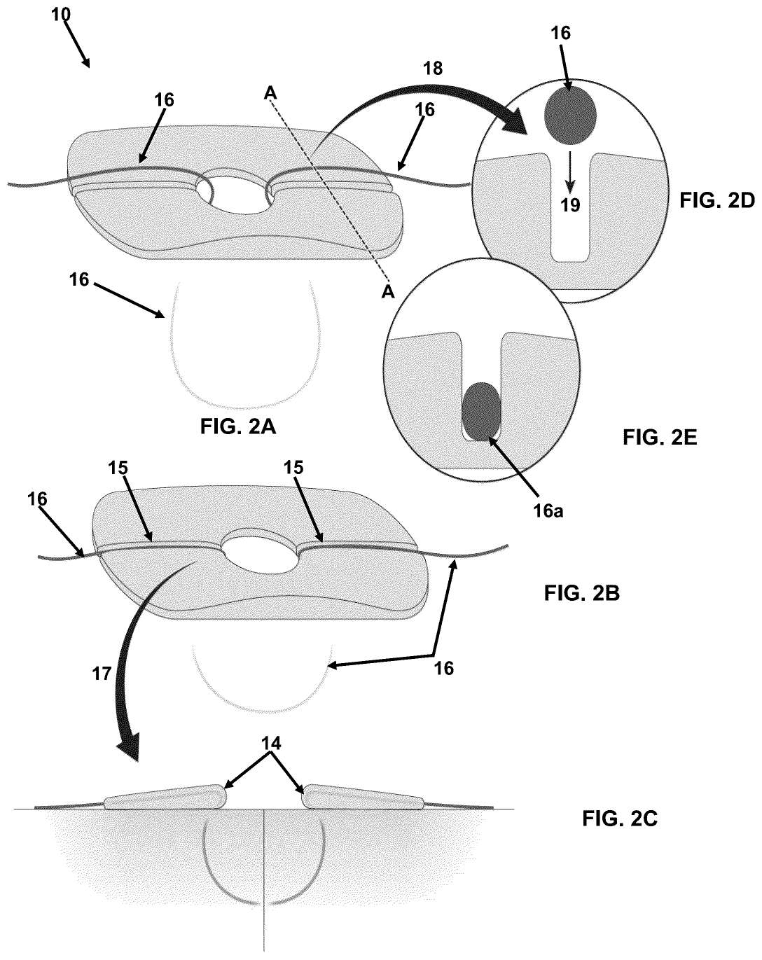

With continued reference to FIGS. 1A-1C, FIGS. 2A-2E illustrate the engagement of the suture within the STITCHLOCK device 10. The suture 16 is threaded through the central aperture 13 prior to engaging with the groove 15. The suture engages within the groove and with the curvature 14 of the central aperture at 17. In cross-section along A-A at 18 the relationship of the flat underside 11 to the convex top side 12 is shown and illustrates how the suture 16 is compressed to suture 16a after engaging the groove along 19.

With continued reference to FIGS. 2A-2C, FIG. 3 illustrates a wound closure via the STITCHLOCK devices. Wound 20 is shown sutured at 20a,b with STITCHLOCK devices 10a,b locking the suture 16 in place. The suture is threaded into and out of the central aperture 13 of the STITCHLOCK device 10c through the wound tissue at 20c. Engaging the suture tautly and securely within the groove 15 via instrument 21 and around the curvature of the aperture 14 will draw the margins of the wound together as shown at 20a,b.

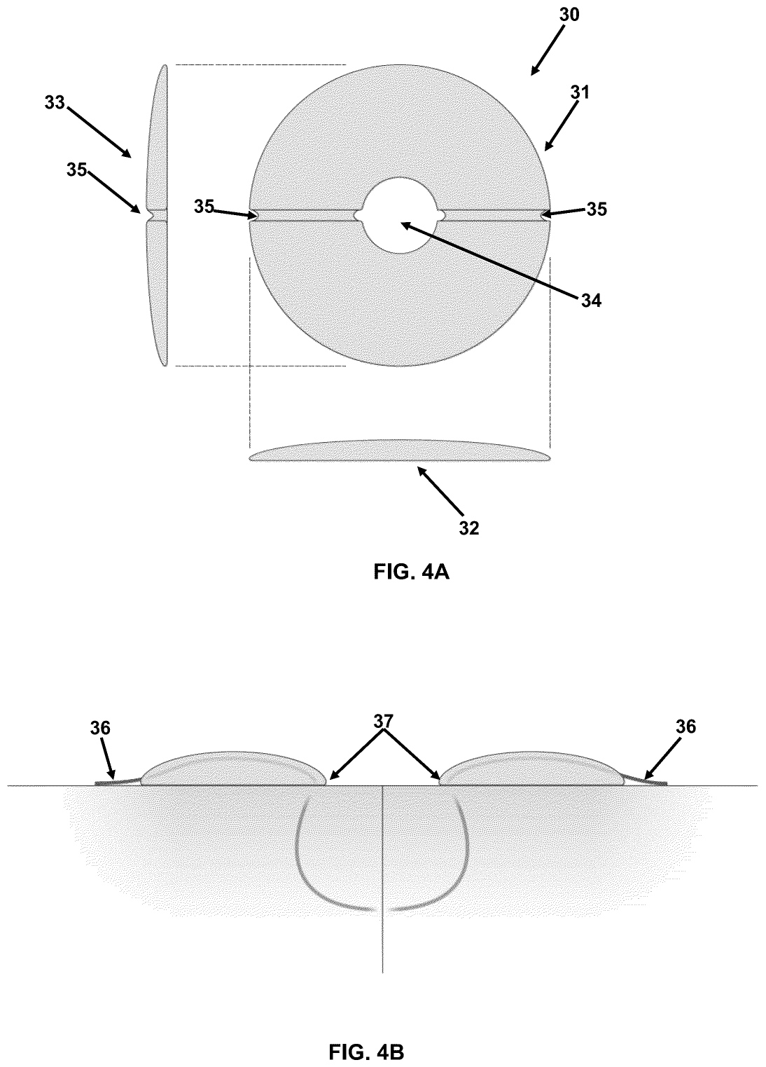

FIGS. 4A-4B illustrate a second embodiment of the STITCHLOCK device 30. The device is circular in overall shape at 31 with a flat underside 32 and a convex top side 33. The device has an aperture 34 and a groove 35 with which to secure a suture 36. The device has a curvature 37 along the aperture that is formed from the flat underside around the aperture and into the groove on the topside.

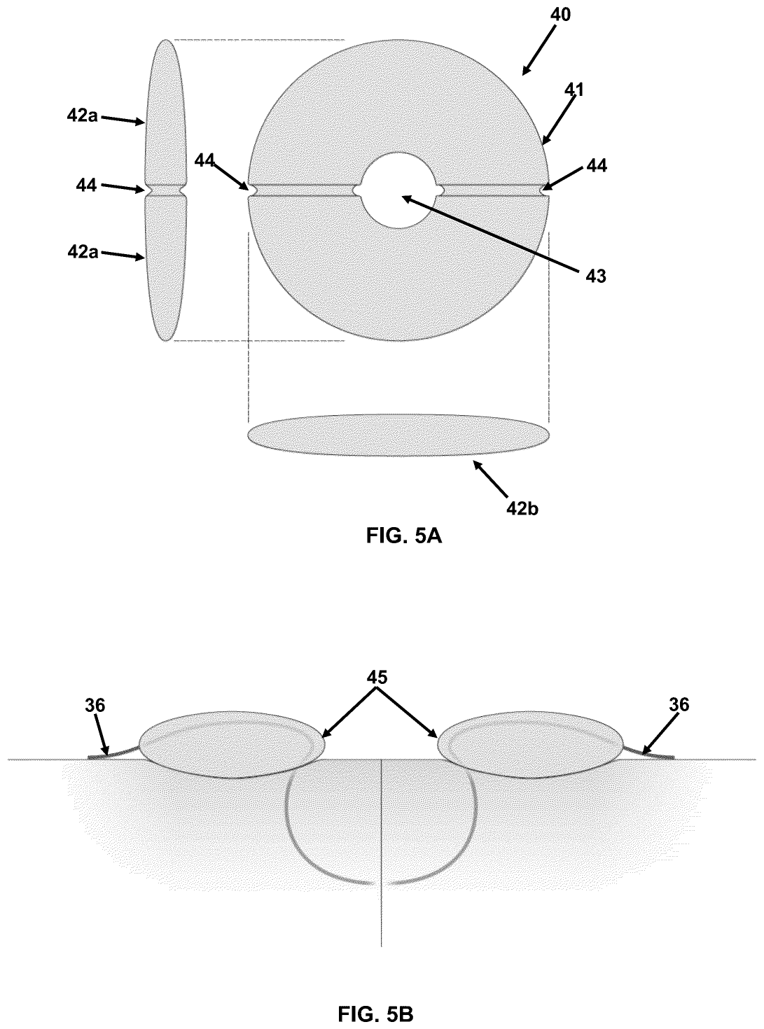

FIGS. 5A-5B illustrate a third embodiment of the STITCHLOCK device 40. As with FIG. 4A the device is circular in overall shape at 41 but with a first convex surface 42a and a second convex surface 42b. The device has an aperture 43 and a groove 44, which is curved where it traverses the aperture at 45, to secure a suture 36. The device does not have differently shaped first and second surfaces which, in view of the identical convexities, enables the surgeon to place the device in either orientation.

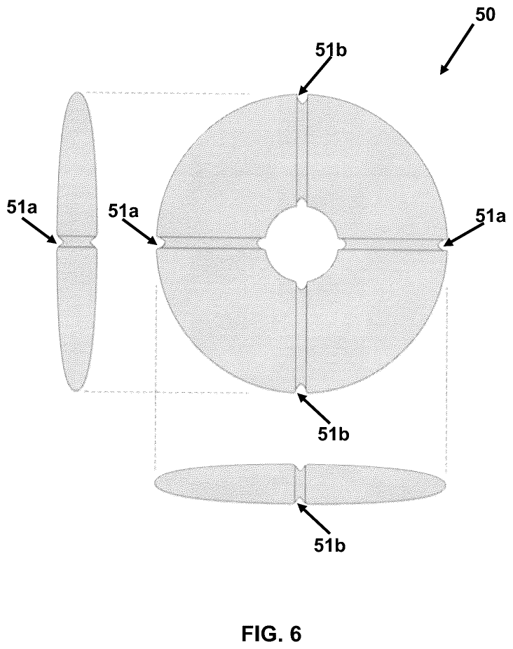

With continued reference to FIGS. 5A-5B, FIG. 6 illustrates a fourth embodiment of the STITCHLOCK device 50. The device differs from the third embodiment in that a first groove 51a and a second groove 51b are positioned 90 degrees relative to one another.

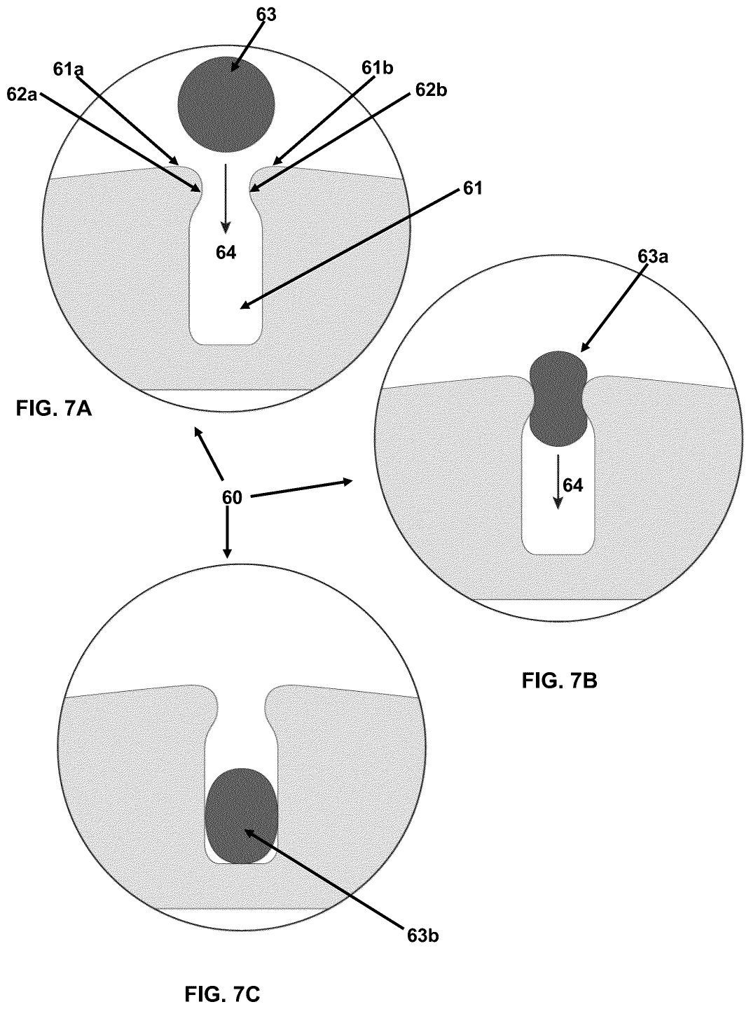

FIGS. 7A-7C illustrate a fifth embodiment of the STITCHLOCK device 60. The groove 61 has shoulders 62a,b at the upper edges 61a,b thereof. The suture 63 is inserted into the groove along 64 and is compressed to 63a by the shoulders. The suture is still compressed 63b within the groove to about 10% to about 60%. The shoulders at upper edges of the groove enable both a haptic feedback to the surgeon signifying engagement of the suture and means for preventing the suture from backing out of the groove.

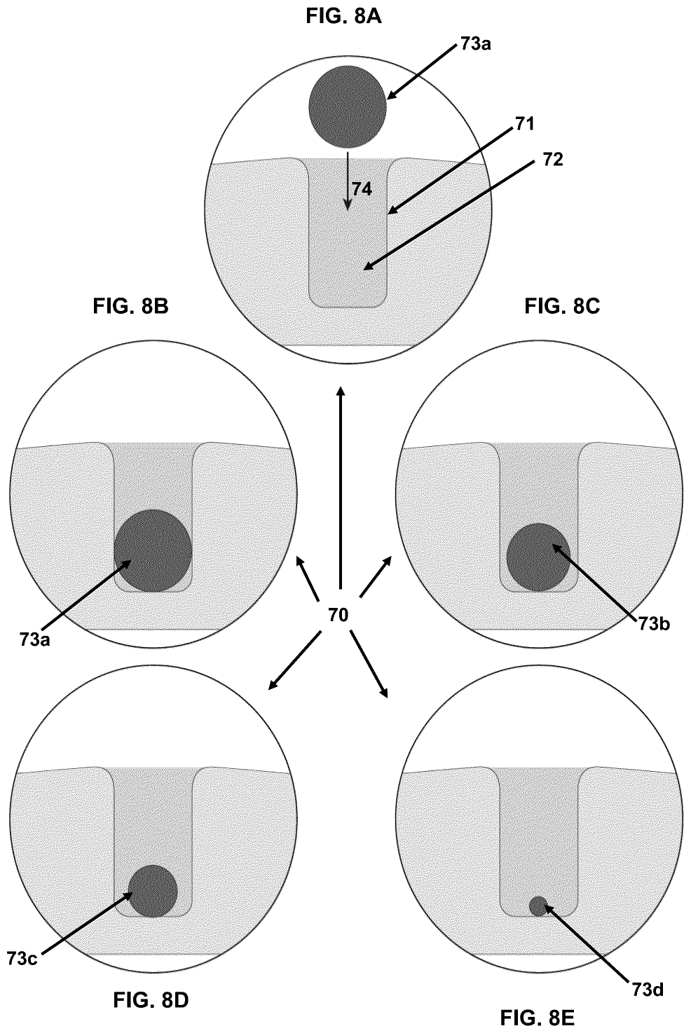

FIGS. 8A-8E illustrate a sixth embodiment of the STITCHLOCK device 70. The groove 71 contains a softer or similarly absorbable polymer 72 than is affixed to the device. The groove does not engage the suture by compression and has a diameter that is equal to or wider than all gauges of suture, represented by 73a,b,c,d. The suture is submerged along 74 into the polymer in the groove where the cumulative tension along the groove prevents the suture from pulling out.

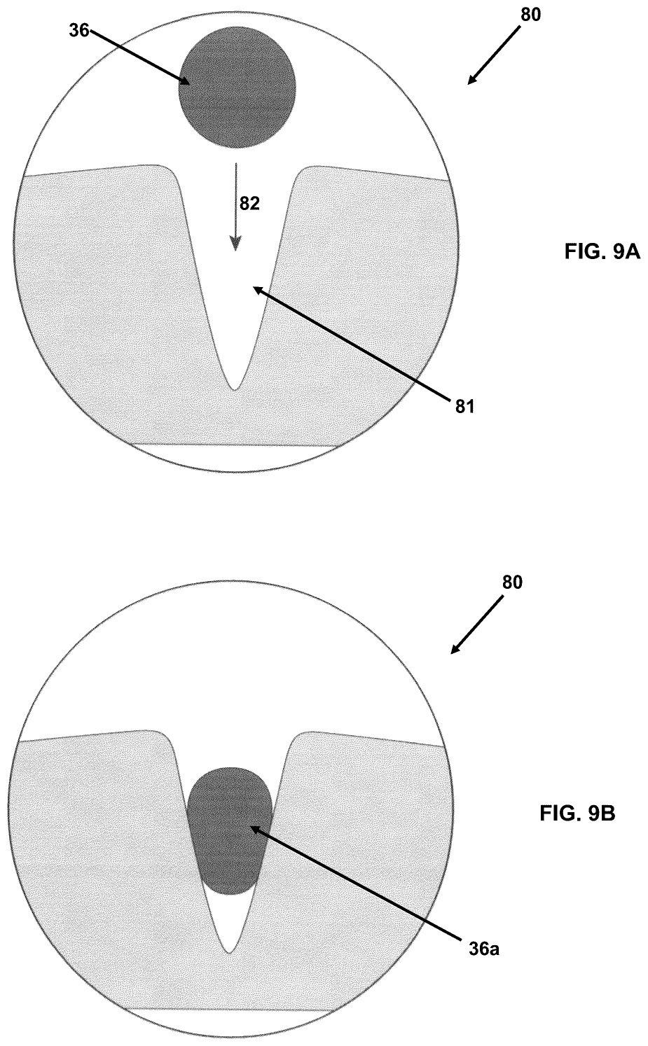

FIGS. 9A-9B illustrate a seventh embodiment of the STITCHLOCK device 80. The groove 81 is V-shaped. The suture 36 is compressed to 36a and engages the groove as the groove tapers along 82.

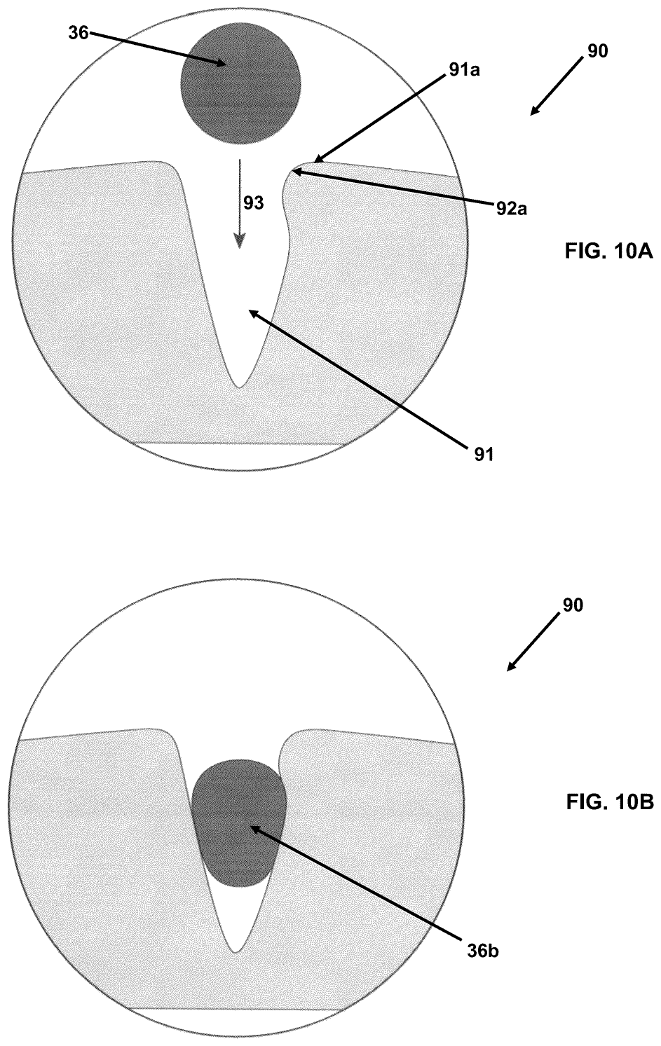

With continued reference to FIGS. 9A-9B, FIGS. 10A-10B illustrate an eighth embodiment of the STITCHLOCK device 90. The groove 91 is V-shaped and has a shoulder 92a at one of the upper edges 91a thereof. The suture 36 is compressed to 36b first by the shoulder and then as the suture engages the groove as the groove tapers along 93.

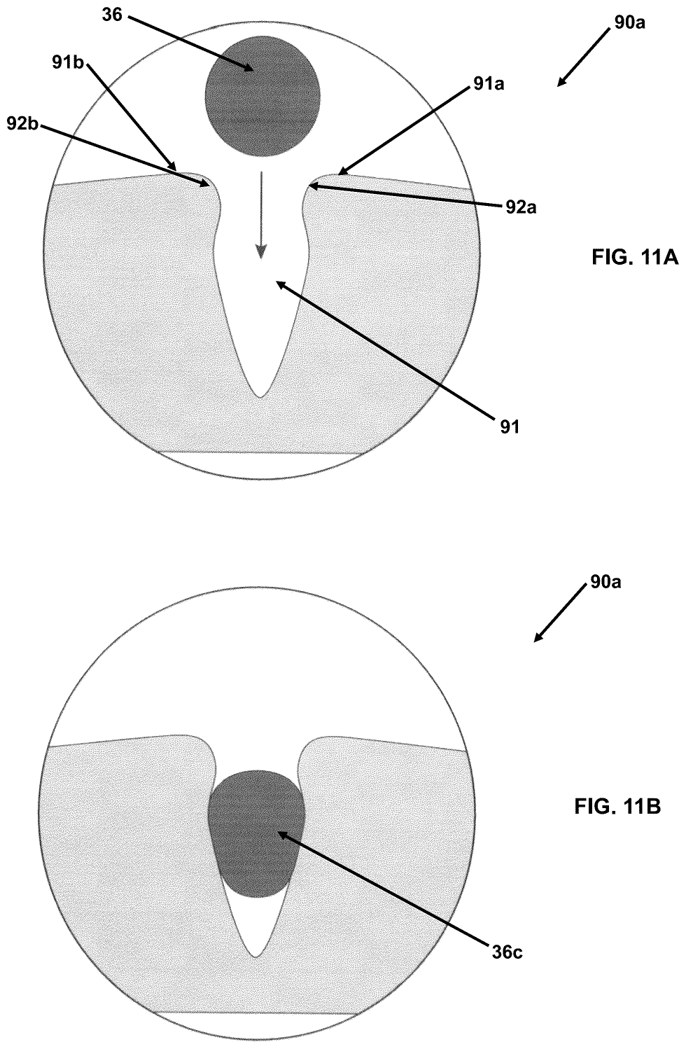

With continued reference to FIGS. 10A-10B, FIGS. 11A-11B illustrate a related ninth embodiment of the STITCHLOCK device 90a. The V-shaped groove 91 has shoulders 92a,b at the upper edges 91a,b thereof. The suture 36 is compressed to 36c first by the shoulders and then as the suture engages the groove as the groove tapers along 93.

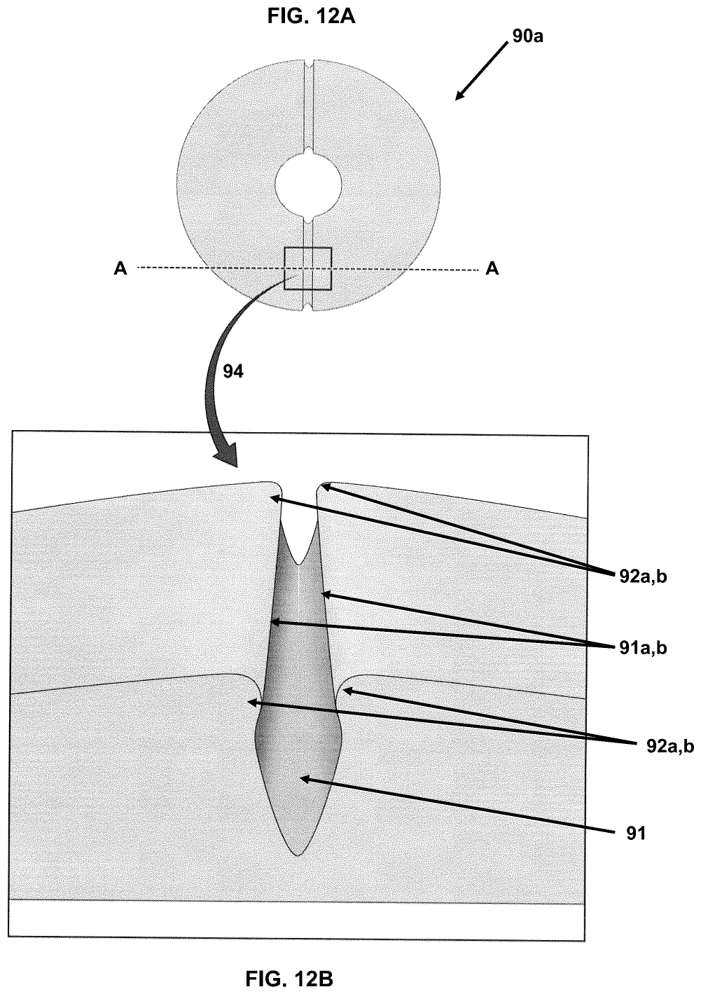

With continued reference to FIG. 11A, FIGS. 12A-12B show a top view of the circular STITCHLOCK device 90a and an enlargement of section 94 on the device shown as a cross-section across A-A. The cross-sectional view 90a better illustrates the V-shape of the groove 91 and the shoulders 92a,b formed along the upper edges 91a,b of the groove 91 across the surface of the device.

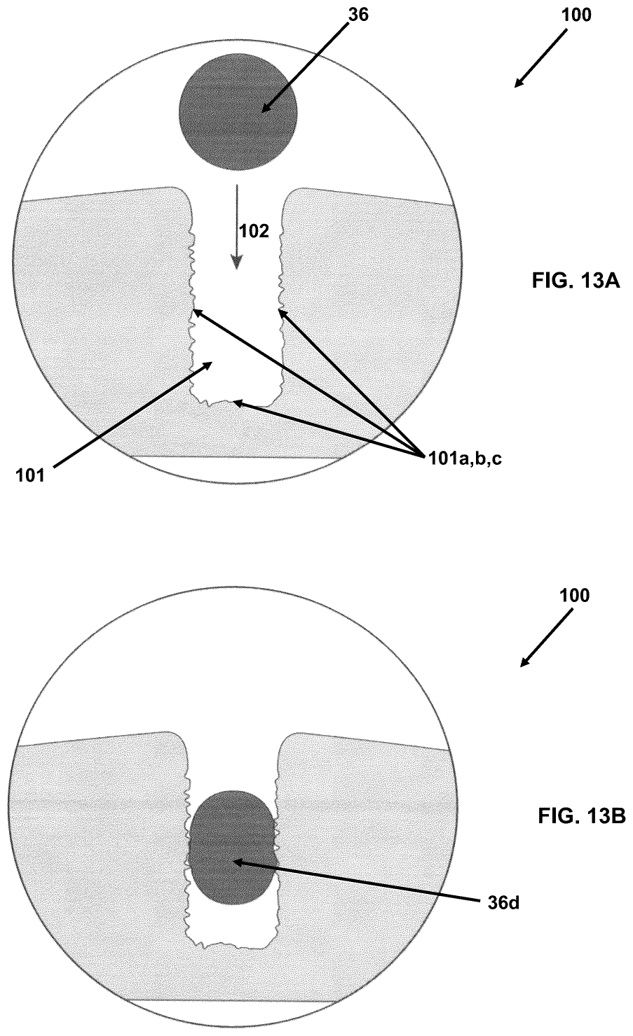

FIGS. 13A-13B illustrate a tenth embodiment of the STITCHLOCK device 100. The groove 101 is a standard groove where the inner surfaces 101a,b,c thereof are roughened. The roughened surfaces provide additional friction to suture 36 as it is compressed by the sidewalls 101a,b along 102.

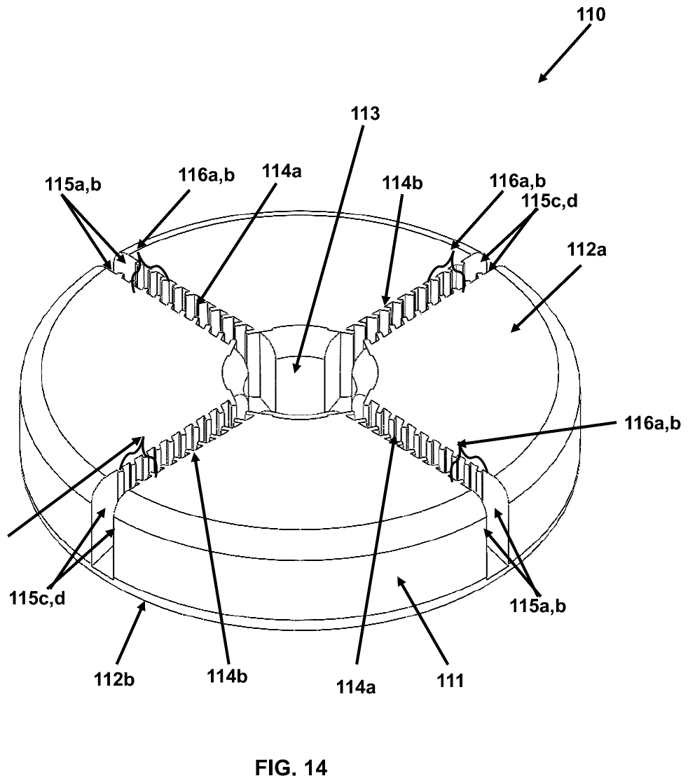

FIG. 14 illustrates an eleventh embodiment of the STITCHLOCK device 110. The device has a substantially circular perimeter at 111 with a substantially flat upper surface 112a and a substantially flat lower surface 112b. As with FIG. 6 the device has a central aperture 113 and a first groove 114a and a second groove 114b are positioned 90 degrees relative to one another. The side surfaces 115a,b of the first groove and the side surfaces 115c,d of the second groove have a plurality of teeth with sharp ends represented by 116a,b and 116c,d. The plurality of the sharp teeth embed themselves into the suture as the surgeon presses the suture into the groove. In this embodiment the wound is transverse to the path of the suture and is located at the central aperture.

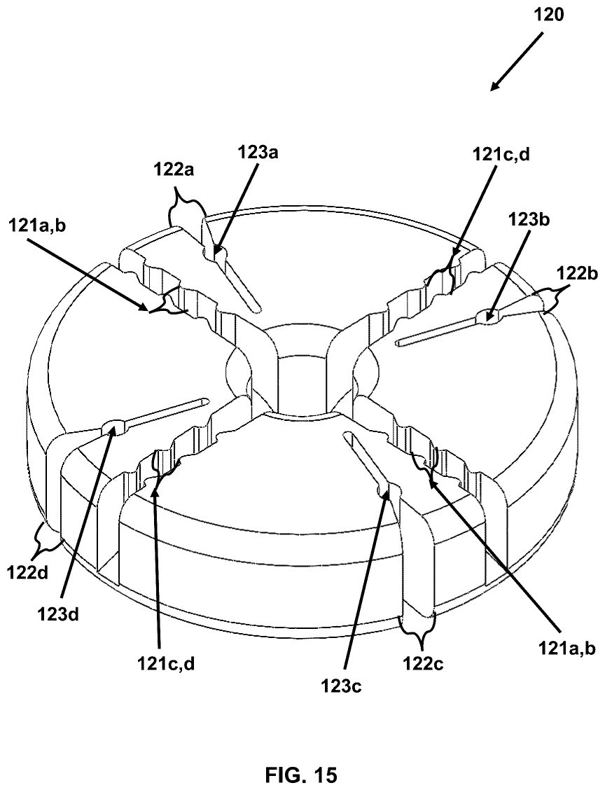

With continued reference to FIG. 14, FIG. 15 illustrates a twelfth embodiment of STITCHLOCK device 120. The device differs from the eleventh embodiment in the number and size of the plurality of teeth with sharp ends represented by 121a,b and 121c,d on the side surfaces of the first groove and of the second groove. This embodiment also comprises slots 122a,b,c,d which are secondary in function to the first groove and to the second groove. The secondary slots have perpendicular openings 123a,b,c,d through which the suture passes whereupon the suture moves and is squeezed tighter in the slot in the radial direction.

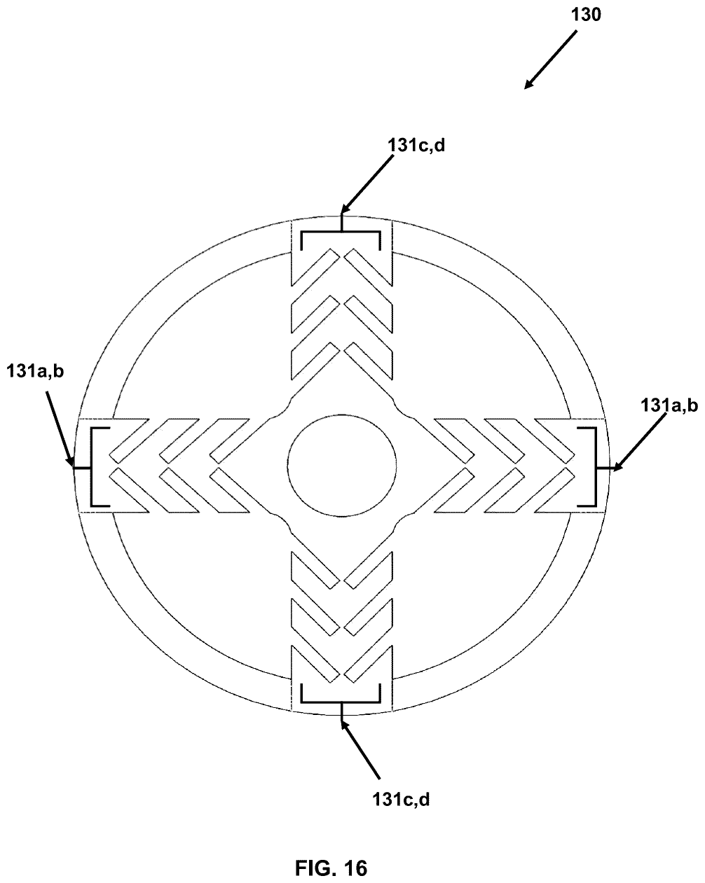

With continued reference to FIG. 14, FIG. 16 illustrates a thirteenth embodiment of the STITCHLOCK device 130. The device differs from the eleventh embodiment in that side surfaces of the first groove and of the second groove have a plurality of flexible fingers 131a,b and 131c,d extending laterally into the interiors of the first and second grooves and angling outwardly toward the circular perimeter. When the suture is pulled from the wound site, the flexibility of the fingers enables an inward flexion and mechanical binding of the suture within the first or second groove to prevent it from being pulled out.

What is claimed is:

1. A device for locking a suture on a wound, comprising:
a body with a substantially geometrical shape;
at least one aperture disposed through the device; and
at least one groove disposed across a top side of the body on either side of the aperture.

2. The device of claim 1, further comprising at least one open-ended slot formed longitudinally into the body proximate to each of the at least one groove on either side of the aperture, said slot having a perpendicular opening through which the suture is received and tightened.

3. The device of claim 1, wherein the body comprises a substantially rectangular shape or a circular shape.

4. The device of claim 3, wherein the body comprises rounded edges at a perimeter or a circumference thereon.

5. The device of claim 1, wherein the body comprises a convex top side and a flat underside.

6. The device of claim 1, wherein the body comprises a convex upper surface and a convex lower surface.

7. The device of claim 1, wherein the aperture is disposed through a center of the device.

8. The device of claim 1, wherein the aperture is curved at a circumference thereof.

9. The device of claim 1, wherein the device comprises one groove.

10. The device of claim 1, wherein the device comprises a pair of grooves disposed at 90 degrees relative to one another.

11. The device of claim 1, wherein the device comprises a plurality of grooves radially disposed on either side of the aperture.

12. The device of claim 1, wherein the groove has a diameter sufficient to secure therein a suture of a specific size.

13. The device of claim 1, wherein the groove has a diameter greater than a diameter of a suture, said groove containing a soft, absorbable polymer.

14. The device of claim 1, wherein the groove has a rectangular shape, has a U-shape or has a V-shape.

15. The device of claim 1, wherein the groove narrows across a width thereof from the aperture to an edge of the device or narrows across the width from the edge of the device to the aperture.

16. The device of claim 1, wherein the groove further comprises one shoulder or a pair of shoulders each disposed along an upper edge of the groove.

17. The device of claim 1, wherein the groove comprises at least one roughened inner surface or a plurality of flexible fingers or a plurality of teeth with substantially sharp ends disposed on at least one inner surface thereof.

18. The device of claim 1, wherein the body is made of an absorbable or non-absorbable polymer.

19. The device of claim 1, wherein the body is color-coded or numbered to correlate to a suture size or suture type or a combination thereof.

20. A stitch locking system, comprising:
the device of claim 1; and
a suture attached by an end thereof to a distal end of the groove.

21. A device for locking a suture on a wound, comprising:
a substantially rectangular body made of an absorbable polymer or a nonabsorbable polymer, said body having a convex top side and an underside with a rounded perimeter thereon, a central aperture therethrough and a substantially rectangular groove disposed longitudinally across the top side on either side of the central aperture; said central aperture comprising a circumferential curvature and said groove comprising a curved shoulder along one upper edge or both upper edges.

22. The device of claim 21, wherein the underside is flat or convex.

23. The device of claim 21, wherein the substantially rectangular groove has a diameter greater than a diameter of each of a plurality of a suture sizes, said substantially rectangular groove containing a soft, absorbable polymer therein.

24. The device of claim 21, wherein the groove has a diameter sufficient to secure therein a suture of a specific size.

25. The device of claim 21, wherein the groove comprises at least one roughened inner surface.

26. A device for locking a suture on a wound, comprising:
a circular body comprising an absorbable polymer or a nonabsorbable polymer, said body having a top side and an underside with a rounded circumferential edge, a central aperture therethrough and a first V-shaped groove disposed longitudinally across the top side on either side of the central aperture; said central aperture comprising a circumferential curvature and said first V-shaped groove comprising a curved shoulder along one upper edge or both upper edges.

27. The device of claim 26, further comprising a second V-shaped groove disposed across the top side on either side of the central aperture at 90 degrees relative to the first V-shaped groove.

28. The device of claim 27, wherein the first V-shaped groove and the second V-shaped groove each have a same dimension or have different dimensions each sufficient to secure therein a suture of a specific size therein.

29. The device of claim 27, wherein the first V-shaped groove or the second V-shaped groove or a combination thereof has a diameter greater than a diameter of a plurality of suture sizes and a soft, absorbable polymer contained therein.

30. The device of claim 26, wherein the underside is flat or convex.

31. A device for locking a suture on a wound, comprising:
a body with a substantially circular perimeter, a substantially flat upper surface and a substantially flat lower surface;
an aperture disposed perpendicularly through the center of the device;
a first groove and a second groove each disposed across the upper surface of the body on either side of the aperture, said first groove and said second groove comprising means for increasing securement of the suture disposed on side surfaces thereof.

32. The device of claim 31, further comprising a secondary slot with an open end at the circular perimeter and extending longitudinally into the body proximate to the first groove and to the second groove on either side of the aperture, said secondary slot having a perpendicular opening through which the suture is received and tightened.

33. The device of claim 31, wherein said means for increasing securement of the suture comprises a plurality of teeth with sharp ends extending interiorly from the side surfaces of the first groove and the second groove.

34. The device of claim 31, wherein said means for increasing securement of the suture comprises a plurality of flexible fingers extending laterally into the interiors of the first and second grooves and angling outwardly toward the circular perimeter.

35. The device of claim 31, wherein the first groove and the second groove independently have a rectangular shape, a U-shape or a V-shape.

36. The device of claim 31, wherein the first groove and the second groove each have dimensions sufficient to secure a suture of a single size therein.

37. The device of claim 31, wherein the first groove and the second groove have dimensions sufficient to secure sutures of different sizes therein.
